(19) United States
(12) Patent Application Publication
(10) Pub. No.: US 2024/0374417 A1
(43) Pub. Date: Nov. 14, 2024

(54) MULTI-FUNCTIONAL HEAT DISTRIBUTION SYSTEM

(71) Applicant: ALPHAPOINTE, Kansas City, MO (US)

(72) Inventors: Jeremy Neskey, Underhill, VT (US); Christopher Kosiorek, La Vernia, TX (US); Ryan Williams, Olathe, KS (US); Brenda Mee, West Newfield, ME (US)

(21) Appl. No.: 18/587,805

(22) Filed: Feb. 26, 2024

Related U.S. Application Data
(60) Provisional application No. 63/461,560, filed on Apr. 24, 2023.

(51) Int. Cl. A61F 7/00; F24H 3/00

(52) U.S. Cl. CPC A61F 7/0085; A61F 7/007; F24H 3/002; F24H 3/008; A61F 2007/006; A61F 2007/0071; F24H 2250/02

(57) ABSTRACT

A multi-functional warming system is provided to accommodate continuous or near continuous heat distribution for trauma patients. The system is designed for initial deployment immediately following injury in the field while facilitating continued use during transit and in medical facilities. The system includes an air distribution system and an electrical resistance system, each system being configured to work independent or in conjunction with each other. The system also accommodates use of chemical heating systems. The air distribution system includes a combustion chamber that is configured to selectively receive a combustion assembly or an electrical assembly, thereby facilitating respective combustion and electrical heat generation methods.

### CROSS-REFERENCE TO RELATED APPLICATIONS

This application claims priority pursuant to 35 U.S.C. 119 (e) to U.S. Provisional Patent Application Ser. No. 63/461,560, filed Apr. 24, 2023, the entire disclosure of which is incorporated herein by reference.

### GOVERNMENT RIGHTS

This invention was made with government support under HQ0034-20-2-0007 awarded by the U.S. Department of Defense. The government has certain rights in the invention.

### FIELD OF THE INVENTION

The present invention relates generally to heat distribution systems. More specifically, the present invention is concerned with a personal heat distribution system that is capable of using multiple heat generation methods.

### BACKGROUND

Hypothermia, acidosis, and coagulopathy constitute the “triad of death” in trauma patients. The association of hypothermic coagulopathy with increased mortality has been well documented. As many as 66% of trauma patients arrive in emergency departments manifesting some degree of hypothermia (temperature <96.8° F. or 36° C.). Over 80% of non-surviving patients have had a body temperature of less than 34° C. This degree of hypothermia causes dysfunction of coagulation proteins, thus exacerbating hemorrhage and increasing acidosis, which in turn leads to decreased heart performance. Left unchecked, decreased heart performance can lead to loss of body temperature, thereby putting the patient in further risk. With the above in mind, it is no surprise that the mortality in combat casualties with hypothermia is double that of normothermic casualties with similar injuries. Accordingly, it would be beneficial to have a heat distribution system that is capable of maintaining a patient's body to prevent hypothermic coagulopathy.

Prevention of hypothermia must be emphasized in combat operations and casualty management at all levels of care. Hypothermia occurs regardless of the ambient temperature; hypothermia can, and does, occur in both hot and cold climates. Because of the difficulty, time, and energy required to actively re-warm casualties, significant attention must be paid to preventing hypothermia from occurring in the first place. Prevention of hypothermia is much easier than treatment of hypothermia; therefore, prevention of heat loss should start as soon as possible after the injury. Accordingly, it would be beneficial to have a heat distribution system that can be quickly and easily deployed in the field immediately after an injury has occurred.

Currently available “field ready” warming technologies perform well at sea level and ambient temperatures above 0° Celsius. These include systems with an air-activated warming medium and various types of wraps to mitigate heat loss. Unfortunately, these systems tend to lose their effectiveness at elevations above 10,000 feet and in sub-zero temperatures. This can be a major issue when using non-pressurized evacuation platforms at higher elevations and/or when operating in arctic or other low-temperature regions. While some existing systems have increased effectiveness at altitude, such as by using an electrolyte-based water solution for activation of the warming grid when exposed to air, these elevation-effectiveness solutions tend to be ineffective or completely infeasible in sub-zero temperatures. Accordingly, it would be beneficial to have a heat distribution system that is effective in a variety of environments and conditions, including high-elevation environments and low-temperature conditions.

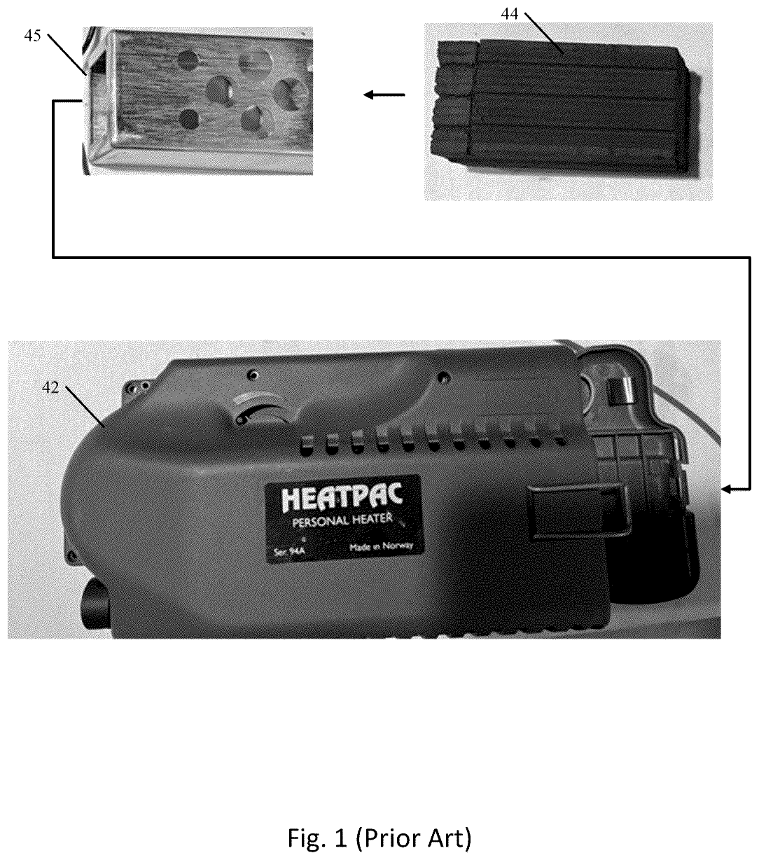

The HEATPAC system shown in FIG. 1, utilizes an outer casing 42 having a combustion chamber that is configured to receive a combustion assembly. The combustion assembly includes a combustion insert 45 and a solid fuel element 44 positioned therein. The combustion insert 45 facilitates retention and combustion of the solid fuel element 44 within the combustion chamber of the outer casing 42. Heat energy generated from the combustion process is utilized to warm air, which the HEATPAC system distributes to an area of need, such as to provide warmth to a person, to warm or dry boots or articles of clothing, or the like. Unfortunately, a solid fuel element 44 may not be available, or supply of such fuel may be limited. Furthermore, some situations do not provide adequate or desired ventilation for the exhaust fumes generated by the combustion process. Accordingly, it would be beneficial to have an additional or alternative means of generating heat energy while utilizing the distribution features of the HEATPAC system.

Severe injuries can be triaged in the field, but they eventually require transportation to a medical facility. Depending on the location, topography, weather conditions, proximity to enemy combatants, and other factors, transportation from the field to a medical facility can be time consuming and may require several different modes of transportation. Upon arriving to a medical facility, patients are often moved from one location to another, and sometimes patients are moved from one medical facility to another. Unfortunately, heating methods that are feasible in the field are not necessarily feasible during transportation or in a medical facility, and heating methods that are feasible for one form of transportation or one location within a medical facility are not necessarily feasible for other forms of transportation or other locations within a medical facility. Accordingly, it would be beneficial to have a heat distribution system that is capable of using various methods of maintaining a patient's body temperature so that it is feasible to maintain the patient's body temperature in the field, throughout transportation to a medical facility, and regardless of where the patient is within the medical facility.

### SUMMARY

The present invention comprises a unique active heat distribution system that is capable of maintaining a patient's body temperature to prevent hypothermic coagulopathy. In some embodiments, the present invention is integrated into or works with a vest, a blanket, or a similar item that can be quickly and easily deployed in the field immediately after an injury has occurred. The heat distribution system of the present invention is effective in a variety of environments and conditions, including high-elevation environments and sub-zero temperature conditions. In some embodiments, the system of the present invention is capable of maintaining a patient's body temperature in the field, in transit, and in a medical facility. In some such embodiments, the system of the present invention utilizes multiple sources of active warming variations thereby increasing versatility and reliability of the system. In some embodiments, the multiple sources of active warming variations include forced hot air (both fuel sourced and electrical), chemical reaction, and electrical resistance heating technology combined into one distribution system. Accordingly, the device of the present invention can be used across the spectrum of the patient care continuum, accepting all sources of fielded technology to continue warming a hypothermic patient from point of injury through hospital care, including all forms of patient transport (air and ground).

Prevention of hypothermia is much easier than treatment of hypothermia; therefore, prevention of heat loss should start as soon as possible after the injury and should be maintained with little to no interruption until the patient's condition is stabilized to the point that heat regulation assistance is no longer required. The system of the present invention is designed to be rugged, lightweight, and durable, thereby enabling the system to be located in the field so that it is located as close as possible to the point of anticipated or potential injury. The present invention is further designed for quick and easy deployment, thereby decreasing the amount of time necessary to begin life-saving heat regulation for a patient. Finally, the present invention is designed to be utilized in the field and at all subsequent levels of care, including ground and air evacuation, across the spectrum of theaters, thereby facilitating constant heat regulation assistance until such assistance is no longer required.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION

Embodiments of the present invention are described in detail below with reference to the attached drawing figures, wherein:

FIG. 1 shows an outer casing and a combustion insert of an existing warming system, as well as solid fuel for the existing warming system, the combustion insert and the solid fuel shown removed from the outer casing of the existing warming system.

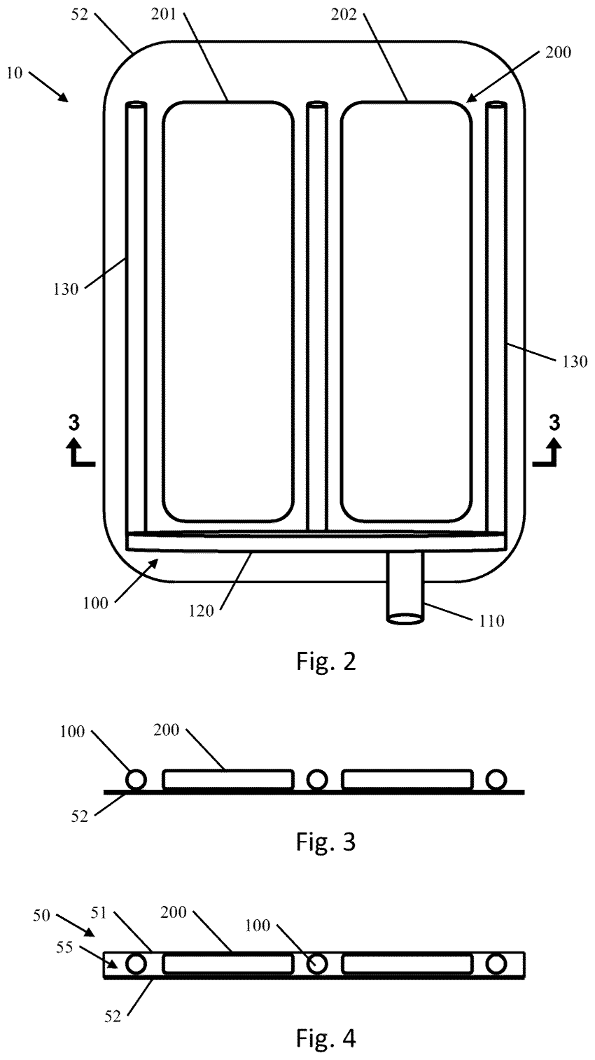

FIG. 2 is a schematic view of a multi-functional warming system of the present invention.

FIG. 3 shows the cross section of the multi-functional warming system of FIG. 1 taken from line 3-3 of FIG. 2.

FIG. 4 shows a cross section of an alternative embodiment of the multi-functional warming system of the present invention.

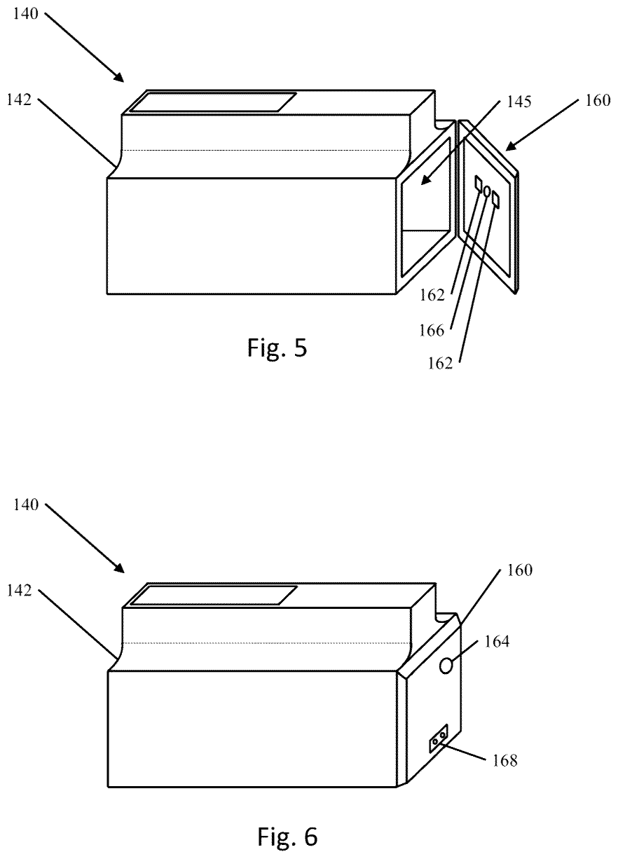

FIG. 5 shows an air distribution system of the present invention, the system shown in an open configuration.

FIG. 6 shows an air distribution system of the present invention, the system shown in a closed configuration.

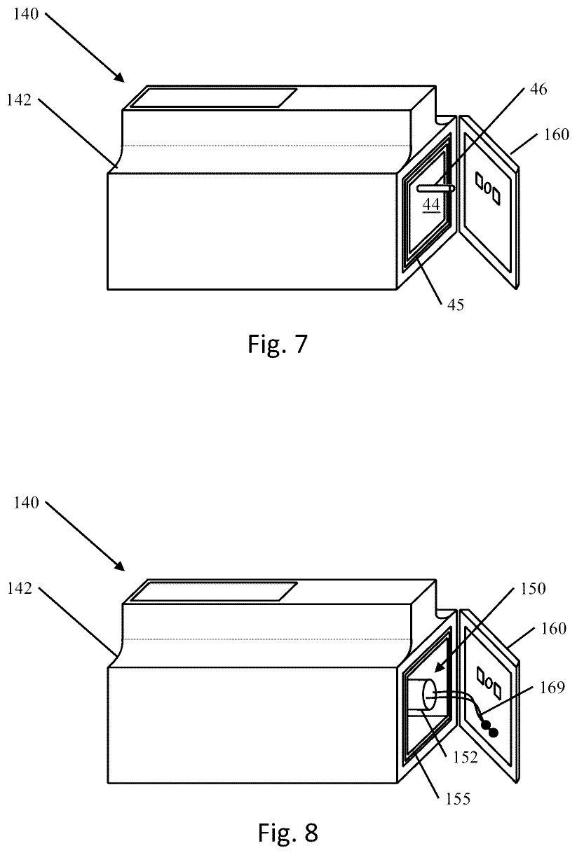

FIG. 7 shows an air distribution system of the present invention, the system shown in an open configuration with a combustion insert and a solid fuel element positioned within an inner area of the system.

FIG. 8 shows an air distribution system of the present invention, the system shown in an open configuration with an electrical assembly positioned within an inner area of the system.

The drawing figures do not limit the present invention to the specific embodiments disclosed and described herein. The drawings are not necessarily to scale, emphasis instead being placed upon illustrating the principles of the invention.

### DETAILED DESCRIPTION

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to FIGS. 2 and 3, the warming system 10 of the present invention includes first 100 and second 200 subsystems for generating and/or distributing heat, each subsystem being secured to a main body 50. The main body 50 is configured to wrap around a torso of a patient, thereby creating a thermal shell around the patient with the first and second subsystems being positioned at least partially within the thermal shell. In some embodiments, the warming system 10 includes form and function similar to a blanket, thereby allowing a user to place the warming system over, under, or around a patient, as needed. In other embodiments, the warming system 10 includes form and function similar to a vest or other article of clothing, thereby allowing a user to engage the warming system 10 in a manner that is readily apparent and understandable while also being extremely secure and reliable.

Referring to FIG. 4, some embodiments of the main body include a heat distributing inner layer 51 and a heat retaining outer layer 52 displaced therefrom, thereby forming a cavity 55 within which at least part of the first 100 and second 200 subsystems are positioned. In some embodiments, the cavity 55 is filled at least partially with a thermal medium, the thermal medium being selected for its heat distribution and/or heat retaining properties.

In the embodiment shown, the first subsystem 100 is a forced air distribution system having an inlet valve 110, a single trunk 120 extending in two directions from the inlet valve 110, and a plurality of branches 130 extending from the trunk 120, each branch 130 being displaced from each of the other branches so as to facilitate air distribution over a large area, the large area generally correlating with the core of the patient. The inlet valve 110 is configured to engage with a forced air unit such that air from the forced air unit can be directed through the inlet 110 and the trunk 120 to each of the branches. In some embodiments, the forced air unit generates heat through active burning of fuel, using electrical resistance, through chemical reaction, or other ways of generating heat that are now known or later developed. In some embodiments, each branch 130 of the forced air distribution system defines a plurality of pinholes directing airflow into the thermal shell around the patient, thereby creating a positive pressure environment within the thermal shell.

In some embodiments, an air distribution system 140 of the of the first subsystem 100 includes an outer casing 142 having an inner area 145, such as a combustion chamber or the like. In some such embodiments, the inner area is configured to receive and retain a combustion insert 45 and a solid fuel element 44 of the HEATPAC system of the prior art, thereby facilitating heat distribution during combustion of the solid fuel element 44.

Referring to FIG. 8, some embodiments of the air distribution system 140 are configured to receive an electrical assembly 150. The electrical assembly includes a heating element 152 for converting electrical energy to heat energy. In some embodiments, the heating element 152 is engaged with a positioning insert 155 to facilitate positioning and retaining the heating element 152 within the inner area of the outer casing 142. In some embodiments, the positioning insert 155 includes features and dimensions similar to those features and dimensions of a combustion insert 45 of a traditional HEATPAC system, thereby facilitating selective use of electrical and combustion heating processes. In some embodiments, the positioning insert 155 and/or an improved combustion insert 45 of the present invention is formed from folded sheet material, thereby facilitating ease of production over traditional die cast combustion inserts 45 of the prior art.

The air distribution system 140 of the present invention is reconfigurable to utilize a plurality of heat generation methods. In a first heat generation method, heat energy is produced during a combustion process within an inner area of an outer case 141 of the air distribution system 140. In a second heat generation method, heat energy is produced by providing electrical energy to a heating element 152 positioned within the inner area of the outer case 141. When configured to utilize the first heat generation method, the air distribution system 140 can be reconfigured to utilize the second heat generation method by replacing a combustion assembly with an electrical assembly, and vice versa.

In the embodiment shown, the second subsystem 200 includes first 201 and second 202 pods, each pod being positioned between two branches 130 of the forced air distribution system, thereby enabling the second subsystem 200 to serve as a supplemental and/or alternative heat source over generally the same large area associated with the first subsystem 100.

Referring to FIGS. 5 and 6, a door 160 of the air distribution system 140 is hingedly coupled to the outer casing 142 adjacent to an opening of the inner area 145 of the air distribution system 140. The door 160 moves between open and closed positions, thereby moving the air distribution system between open and closed configurations, respectively. In some embodiments, the door 160 includes an ignition device 162, such as embedded spark diodes or the like. In some such embodiments, the air distribution system includes a trigger device 164, such as a push button embedded on the device. In some embodiments, the trigger device 164 is in communication with the trigger device and/or is otherwise configured to control the ignition device such that triggering the trigger device causes the ignition deice to perform an ignition event, such as creating a spark or the like. In some embodiments, the trigger device is sealed or otherwise protected to reduce corrosion or other detrimental adverse effects associated with exposure of electronic components to environmental conditions.

In some embodiments, the solid fuel element 44 is formulated to be easily ignitable by the ignition device 162 or by other ignition means, such as a match or the like. In some embodiments, the air distribution system is configured to facilitate ignition of the solid fuel while the solid fuel element 44 is positioned in the inner area 145 and the air distribution system 140 is in a closed configuration. In some such embodiments, the solid fuel element 44 includes an ignition mechanism 46, such as a fuse or the like. In some embodiments, the air distribution system defines a retention device 166 for holding the ignition mechanism 46 of the fuel source in close proximity to one or more ignition devices 162. In some such embodiments, the retention device is a one-way insert that is configured to facilitate engagement of a fuse with the insert while preventing or otherwise hindering disengagement of the fuse from the insert. In some embodiments, the insert is configured to retain the fuse between two diodes such that an electrical spark between the diodes will ignite the fuse upon a triggering event associated with the trigger device 164. In this way, the present invention facilitates traditional fuse ignition of the fuel source while maintaining the door in a closed configuration, thereby reducing risk of contaminants getting into the inner area while also reducing risk of debris, heat, or the like from escaping the inner area of the air distribution system. In some embodiments, the triggering event is a two-step process, such as a slide and push action, thereby eliminating or otherwise reducing risk of inadvertent ignition of the fuel source.

In some embodiments, the air distribution system 140 includes a CO2 scrubber, thereby eliminating or otherwise reducing CO2 emissions and the adverse consequences associated therewith. In some embodiments, the scrubber is a soda lime scrubber.

In some embodiments, the door 160 includes an electrical interface 168, such as a 2-prong plug or the like. The electrical interface is configured to connect with an exterior power source, thereby facilitating prolonged use of the heat distribution system when an external power source is available, such as at a medical facility or in transit to the medical facility. In some such embodiments, a heating element 152 of the present invention is in electrical communication with the electrical interface 168, such as through electrical wires 169 or the like. In some embodiments, the present invention includes one or more power adapters, thereby facilitating flexible use of the present invention.

In some embodiments, the heating element 152 includes one or more super-heated pads. In some embodiments, the positioning insert 155 creates a heat sink to facilitate heating of air within the inner area 145 of the present invention. In some embodiments, the heating element 152 is configured to be powered by a number of external power sources, such as a car battery, a cigarette lighter port, direct connections to the electric grid, or the like. In some embodiments, the electrical assembly is configured to be interchangeable with existing traditional solid fuel devices, thereby facilitating retrofitting the same. In some such embodiments, the door 160 of the present invention is also configured to be interchangeable with doors of existing systems that currently are configured only for use with solid fuel, thereby further facilitating retrofitting the same.

In some embodiments, the air distribution system 140 includes one or more inner battery sources for powering features of the device. In some embodiments, the air distribution system 140 includes a waterproof or otherwise water resistant, multi-speed fan and a corresponding multi-position selector switch. In some such embodiment, the fan motor is moveable between an off position, a low (such as 1.5 volts) configuration, and a high (such as 3 volts) configuration.

In some embodiments, the second subsystem 200 is an electrical resistance heat source having a plurality of heat generating resisters positioned within a thermal medium, the thermal medium being selected for its ability to distribute electrical resistive heating while reducing the risk of burning the patient or starting a fire. In some embodiments, the electrical resistance heat source is capable of interfacing with and utilizing power from a plurality of power sources. In some embodiments, the system includes a durable power port that remains with a care provider, the durable power port being configured to quickly and easily engage with an electrical resistance heat source of a warming system 10 of the present invention, each warming system 10 being configured to remain with a respective patient throughout the patient's care.

In some embodiments, the warming system 10 is configured to receive a third heat source, such as a chemical or other heat source, thereby providing additional versatility. In some such embodiments, a main body 50 of the warming system 10 includes pockets or other retention systems to facilitate strategic positioning of one or more chemical pouch or other heat source device. In some embodiments, the second subsystem 200 is a chemical heat source system or is a multi-heat source system that utilizes chemical, electrical, and/or other heat generating methods now know or later developed.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, various embodiments of the present technology include a variety of combinations and/or integrations of the embodiments described herein.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Although the foregoing detailed description of the present invention has been described by reference to one or more exemplary embodiments, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

What is claimed is:

1. An air distribution system for maintaining body temperature of a user in a variety of environments and under a variety of circumstances, the system comprising:
a combustion insert that is configured to facilitate generating heat using a first heat generation process; and
a removable electrical assembly that is configured to generate heat using a second heat generation process,
wherein the first heat generation process is different from the second heat generation process.

2. The system of claim 1, wherein the first heat generation process comprises combustion, and wherein the second heat generation process comprises electrical resistance.

3. The system of claim 2, wherein the first heat generation process utilizes a solid charcoal fuel.

4. The system of claim 1, further comprising a outer casing defining an inner area within which the heat generation of the first and second heat generation processes occur, air within the inner area being heated by the current heat generation process for subsequent distribution to a user.

5. The system of claim 4, further comprising a door hingedly coupled at an opening to the inner area, the door being moveable between open and closed positions, thereby moving the system between open and closed configurations, respectively.

6. The system of claim 5, wherein moving the system to its open configuration facilitate replacement of the combustion insert with the removable electrical assembly, and vice versa, thereby facilitating changing the system between the first and second heat generation processes.

7. The system of claim 6, further comprising a retention device positioned between two electrical diodes, the retention device being a one-way insert for holding a fuse of a solid fuel source between two electrodes of the present invention, the two electrodes being configured to ignite the fuse during an ignition event while the system is in its closed configuration.

8. The system of claim 7, further comprising a trigger device for initiating a trigger event, the trigger device being accessible by a user while the system is in its closed configuration.

9. The system of claim 8, wherein initiating a trigger event is a two-step process, the two-step process comprising a slide action and a push action.

10. The system of claim 9, further comprising directing heated air through tubes of a mobile warming system, the mobile warming system itself comprising a secondary subsystem that is capable of generating heat independent of the air distribution system of the present invention.

11. The system of claim 9, further comprising an electrical interface that is accessible from an exterior of the device, the electrical interface being positioned on the door of the present invention and being configured to interface with a plurality of electrical power sources.

12. The system of claim 11, further comprising a CO2 scrubber, the CO2 scrubber being a soda lime scrubber

13. A method of maintaining body temperature of a user, the method comprising:
utilizing a heat distribution system to generate heat energy through a first heat generation process;
utilizing a mobile warming system to thermally transfer to the user at least some of the heat energy of the first heat generation process;
reconfiguring the heat distribution system to generate heat using a second heat generation process;
utilizing the heat distribution system to generate heat energy through the second heat generation process; and
utilizing the mobile warming system to thermally transfer to the user at least some of the heat energy of the second heat generation process,
wherein the first heat generation processes is a combustion process, and
wherein the second heat generation process is an electrical resistance process.

14. The method of claim 13, further comprising switching from the first heat generation process to the second heat generation process after circumstances for the user change from a first circumstance to a second circumstance, wherein the first circumstance comprises the user being in an open-air environment, and wherein the second circumstance comprises the user being positioned near a reliable source of electrical energy.

15. The method of claim 14, wherein switching from the first heat generation process to the second heat generation process comprises retaining the mobile warming system in position relative to the user, thereby minimizing heat loss from the user.

16. The method of claim 15, utilizing the air distribution system of claim 3.

17. The method of claim 15, utilizing the air distribution system of claim 7.

18. The method of claim 15, utilizing the air distribution system of claim 9.

19. A method of refurbishing an existing air distribution system to convert the system from a single-heating process system to a multi-process system, the method comprising:
removing an original door from an outer casing of the system;
removing a combustion insert from an inner area of the system, the combustion insert being configured to facilitate combustion of a solid fuel source within the inner area of the system;
installing a replacement door onto the outer casing of the system such that moving the replacement door between open and closed positions moves the system between open and closed configurations, respectively, wherein the replacement door comprises an electrical interface for facilitating flow of electrical current into the inner area of the system is in the closed configuration; and
providing an electrical assembly as an alternative heat generation source, the electrical assembly comprising a heating element and a positioning insert,
wherein the electrical assembly and the combustion insert are interchangeable, thereby creating a multi-process system.

20. The method of claim 19, wherein the door comprises two electrodes and a retention device positioned therebetween, the retention device being a one-way insert for holding a fuse of a solid fuel source between the two electrodes, the two electrodes being configured to ignite the fuse during an ignition event while the system is in its closed configuration.
